(19) United States
(12) Patent Application Publication
(10) Pub. No.: US 2021/0229063 A1
(43) Pub. Date: Jul. 29, 2021

(54) AN IMPROVED LED BASED PHOTOCHEMICAL REACTOR

(71) Applicant: PIRAMAL PHARMA LIMITED, Mumbai (IN)

(72) Inventors: Tushar PATEL, Ahmedabad, Gujarat (IN); Sharadsrikar KOTTURI, Ahmedabad, Gujarat (IN); Sasikumar KUTTAPPAN, Ahmedabad, Gujarat (IN)

(73) Assignee: PIRAMAL PHARMA LIMITED, Mumbai (IN)

(21) Appl. No.: 17/264,597

(22) PCT Filed: Aug. 19, 2019
(86) PCT No.: PCT/IB2019/056969

§ 371 Date: Jan. 29, 2021

(30) Foreign Application Priority Data
Aug. 20, 2018 (IN) 201821031193

(51) Int. Cl. B01J 19/12; B01J 8/08; B01J 8/10; C07F 5/02; B01J 31/18

(52) U.S. Cl. CPC B01J 19/122; B01J 8/087; B01J 2531/827; C07F 5/025; B01J 31/1815; B01J 8/10

(57) ABSTRACT

The present invention provides an improved photochemical rector assembly device, particularly a light emitting diode (LED) based small photochemical reactor and methods for performing the photochemical transformations using the instantly presented device. Accordingly, the present invention relates to an improved photochemical transformation reaction by exposing the reaction mixture to a photochemical rector device as shown in fig. A-G, comprising of (i) light emitting diode (LED) panel (1), (ii) Aluminium based heat sink, and (iii) cooling fan.

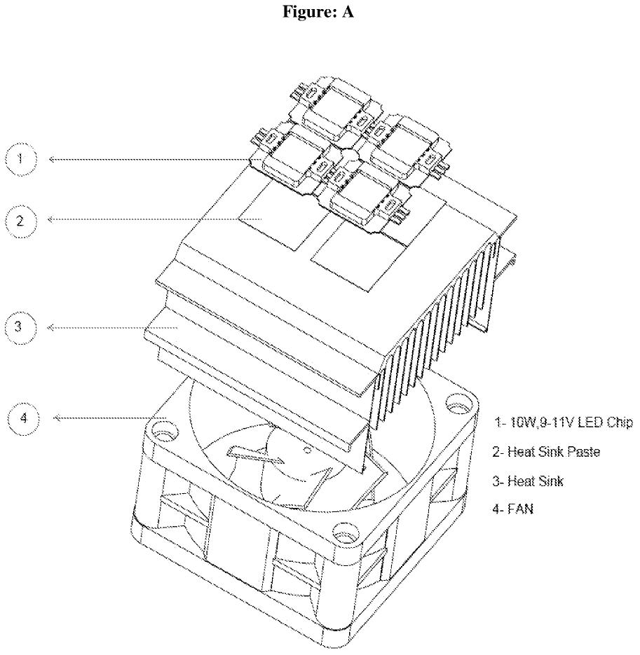

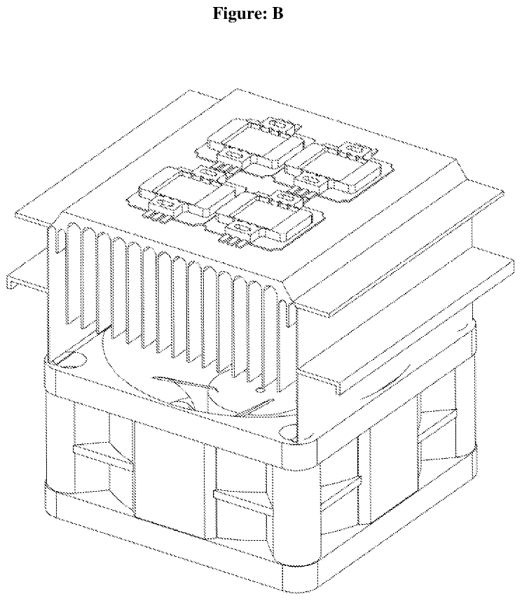

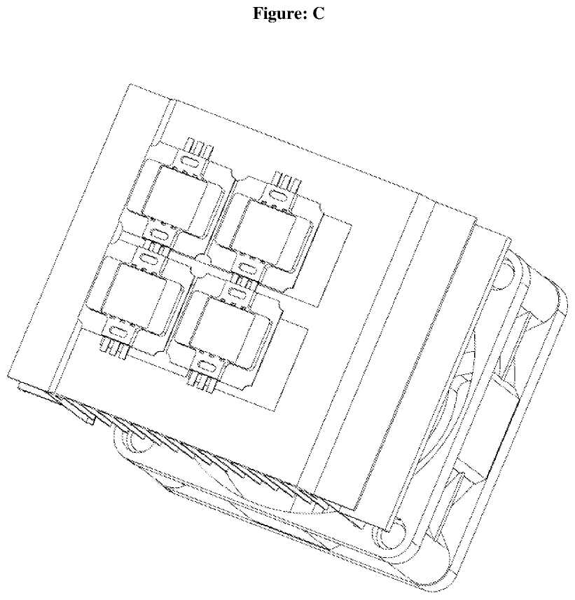

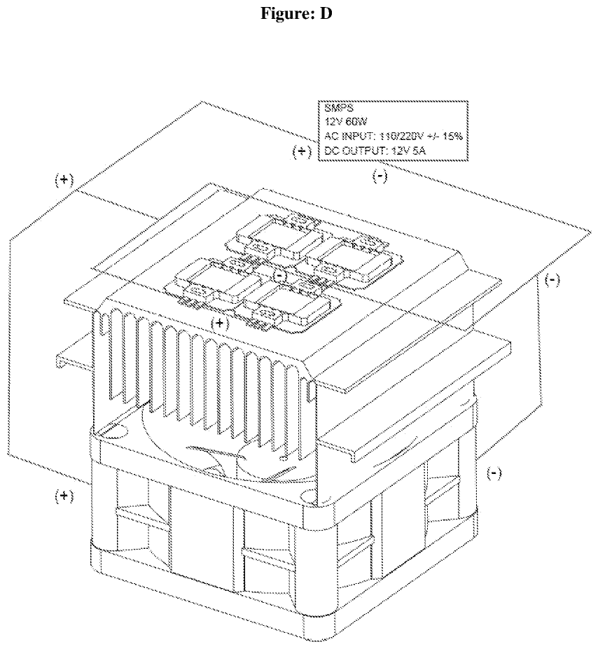

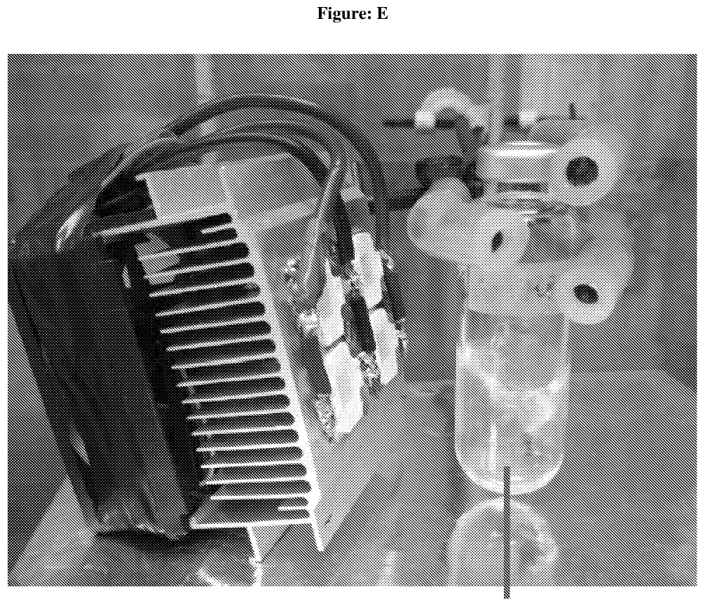

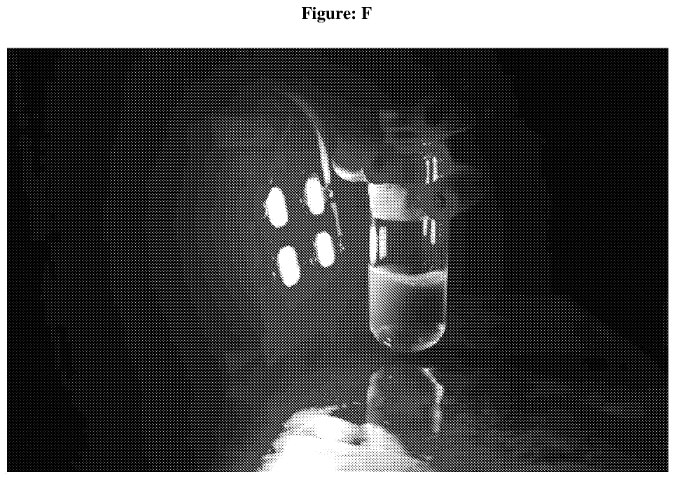

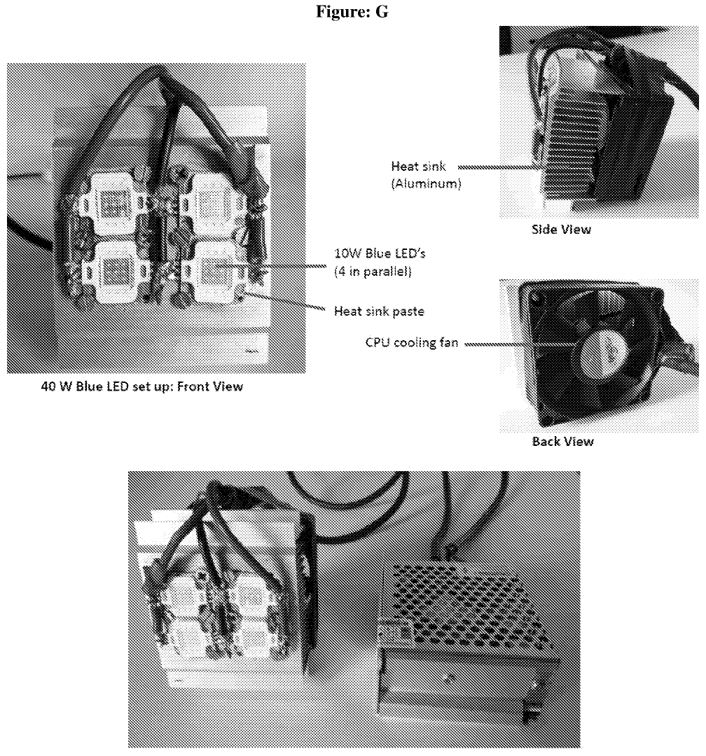

### FIELD OF THE INVENTION

The present invention relates to an improved photochemical rector assembly device, particularly a light emitting diode (LED) based small photochemical reactor and methods for performing the photochemical transformations using the instantly presented device.

### BACKGROUND OF THE INVENTION

The following discussion of the prior art is intended to present the invention in an appropriate technical context, and allows its significance to be properly appreciated. Unless clearly indicated to the contrary, reference to any prior art in this specification should not be construed as an expressed or implied admission that such art is widely known or forms part of common general knowledge in the field.

The photochemical transformations or synthetic photochemistry is a widely followed technology by laboratory scientists, for one of the reason that the photochemical reactions are a ‘green’ alternative to thermal processes or chemically catalyzed reactions. In the photochemistry, chemical reactions are initiated by light, wherein the energy in the form of light is absorbed or emitted by matter leads to an electronic excitation.

Ultraviolet light emitting diodes (UV LEDs) are gaining utmost attention among the researchers to effectively streamline the photochemical transformation. The photochemical transformations, being an important domain in chemistry, a number of photo-catalytic processes as well as photochemical reactors are known in the art.

US patent application No. 2017/0173553 refers to a modular photochemical flow reactor system comprises a plurality of fluidic modules each having i) a central planar process fluid layer and ii) two outer planar thermal control fluid layers for containing flowing thermal control fluid and a plurality of illumination modules. The illumination modules of said plurality each having a planar form with first and second major surfaces and each comprising at least a first array of semiconductor emitters, said emitters positioned to emit from or through the first major surface, wherein said first array of semiconductor emitters comprises at least a first emitter and a second emitter, the first emitter capable of emitting at a first center wavelength and the second emitter capable of emitting at a second center wavelength, said first and second center wavelengths differing from each other. The assembly disclosed in the US'553 is depicted below:

US patent application No. 2009/0143588 disclosed a quartz glass micro-photoreactor as well as synthesis of 10-hydroxycamptothecin and 7-alkyl 10-hydroxycamptothecin using the micro-photoreactor. The quartz glass micro-photoreactor consists of the reactor unit R and two separately closed-off irradiation units (source of radiation). The reactor unit R consists essentially of two quartz glass plates P1 and P2. The gap depth S is defined by a separator which is also made of quartz glass. The quartz glass plates P1, P2 and the separator are connected flush with each other. The fluid stream flows at a flow rate v2 through a bore and can pass through an entry canal before it reaches the irradiated zone. Each irradiation unit (source of radiation) consists of an Fig high pressure emitter (Heraeus Noblelight) and a spectral filter with a specific band pass and a coating on both sides such that the UV emission wavelength range of 350 nm to 400 nm is reached. The output of the Hg high pressure emitter is 500 W. The Hg emitter can also be coated with metal halide ions. The assembly disclosed in the US'588 is depicted below:

Similarly, U.S. Pat. No. 9,938,165 disclosed a reactor that operates with ultraviolet light emitting diodes (UV-LEDs) to attain UV photoreactions or UV photo-initiated reaction in a fluid flow for various applications, including water purification. The UV-LED reactor is comprised of a conduit means for passing fluid flow, an ultraviolet light emitting diode (UV-LED), and a radiation-focusing element to focus the UV-LED radiation to the fluid in the longitudinal direction of the conduit. The UV-LED reactor may include photocatalysts or chemical oxidants, which are activated by UV emitted by UV-LEDs for photocatalytic and photo-initiated reactions. The device disclosed in the US'165 is depicted below:

Also, Brazilian patent application No. 202012000227 disclosed a photochemical reactor having a reaction chamber comprising housing provided with a cover, and cylindrical shaped reaction chamber is provided with a plate to accommodate eight sets of light emitting diode of different emission bands. The light emitting diode is distributed in four vertical columns with ten light emitting diodes equidistant from 90° inside the cylindrical reaction chamber. A transparent container vessel for the reagent is provided for the photochemical reaction resulting from the action of the light emitting diode. The photochemical reactor comprises an electronic interface card for human interface. The reactor comprises a liquid crystal display, a digital encoder, a start button, a micro-controlled electronic control board, integrated circuits, a microcontroller, an elec. power supply and temperature sensor. The device disclosed in the BR'227 is depicted below:

Published Chinese patent application No. 104154455 disclosed LED belt reactor and photo-chlorination reactions using the device. The device comprises LED reaction lights, precisely the LED light belt includes LED lamp and the circuit board, lamp holder includes a cylindrical base body and a snap. The LED light belt is evenly distributed on outer wall of cylindrical seat body via snap. Also, the front end cover is opened with air inlet and hub port and front end cover is fixedly provided at the front end of the lamp holder. The LED light belt is connected with external circuit through a hub port and the center of rear end cover is fixedly provided an rear end of lamp holder. The edge of the front end cover and the rear end cover are provided with embossment, to facilitate the smooth discharge of hot air from the reaction light socket in reactor, to make LED lamp in lower reaction temperature. The device disclosed in the CN'455 is depicted below:

In addition to the aforediscussed patent documents, there are a number of patent documents that describe a photochemical reactor assembly as well as its implementation in the photochemical transformations. For instance, published PCT application WO2004009318; published US patent application 2002/0192569; Chinese patent applications CN103357364, CN105833815, and Japanese patent JP5359063 describes a photochemical reactor and its use in the synthetic photochemistry.

It is evident from the discussion of the photo reactors as well as photochemical transformation using the photo rector assembly, described in the afore cited patent documents that the reported photo-reactors requires specifically designed assembly which comprises inward and outward flow of reaction mixture; hence the reported reactors requires controlled reaction conditions. It is evident that the continuous exposure of the reaction mixture to LED source increases the reaction temperature; however the reactors available in the art requires costlier heat dissipation system such as water circulation cooling system. Also, the reported reactors require comparatively high maintenance cost because the LEDs are placed in a closed system reactors, hence the cleaning and repair of the device requires dismantling of this multipart device and reassembly; hence are not industrially feasible. In view of these drawbacks, there is a need to develop an industrially and economically viable device for the effective photochemical transformation reaction; which is a simple, efficient and cost-effective process and provides the desired reaction product in improved yield and purity.

Inventors of the present invention have developed an improved photochemical reactor device assembly that addresses the problems associated with the devices reported in the prior art and their practical limitations while performing the reactions. The device of the present invention does not involve use of any specialized design and/or costly instrumental parts, also does not involve use of any high cost metal pots and chambers.

Moreover, the process does not require repetitive maintenance, repair or cleaning of the device. Accordingly, the present invention provides a photochemical reactor and its implementation in the photo chemical transformation reaction, which is simple, efficient, cost effective, environmentally friendly and commercially scalable.

### SUMMARY OF THE INVENTION

In one aspect, the present invention relates to a photochemical rector device as shown in fig. A-G; comprising of a light source (1) (as described herein), a heat dissipation assembly (3) (as described herein) and a cooling assembly (4) (as described herein).

In one aspect, the present invention relates to a photochemical rector assembly device as shown in fig. A-G, comprising of (i) light emitting diode (LED) panel (1) (as described herein), (ii) Aluminium based heat sink (3) (as described herein), and (iii) cooling fan (4) (as described herein).

In one aspect, the present invention relates to an improved photochemical transformation reaction by exposing the reaction mixture to a photochemical rector device as shown in fig. A-G, comprising of a light source (1) (as described herein), a heat dissipation assembly (3) (as described herein) and a cooling assembly (4) (as described herein).

In one aspect, the present invention relates to an improved photochemical transformation reaction by exposing the reaction mixture to a photochemical rector device as shown in fig. A-G, comprising of (i) light emitting diode (LED) panel (1) (as described herein), (ii) Aluminium based heat sink (3) (as described herein), and (iii) cooling fan (4) (as described herein).

In another aspect, the present invention relates to an improved photocatalytic sp³-sp² (Carbon-Carbon) coupling reaction by exposing the reaction mixture to a photochemical rector device as shown in fig. A-G, comprising of (i) light emitting diode (LED) panel (1) (as described herein), (ii) Aluminium based heat sink (3) (as described herein), and (iii) cooling fan (4) (as described herein).

In another aspect, the present invention relates to an improved photocatalytic Buchwald type (C-N) coupling reaction by exposing the reaction mixture to a photochemical rector device as shown in fig. A-G, comprising of (i) light emitting diode (LED) panel (1) (as described herein), (ii) Aluminium based heat sink (3) (as described herein), and (iii) cooling fan (4) (as described herein).

### BRIEF DESCRIPTION OF THE DRAWINGS

Various aspects of the invention will be further explained with reference to embodiments shown in the drawings wherein:

Figure: A—is a schematic diagram of the LED device with labelling; constructed and arranged in accordance with the present invention.

Figure: B—is a main view of the LED device represented by the schematic diagram constructed and arranged in accordance with the present invention.

Figure: C—is a top view of the LED device represented by the schematic diagram constructed and arranged in accordance with the present invention.

Figure: D—is a main view of the LED device with electric circuit represented by the schematic diagram constructed and arranged in accordance with the present invention.

Figure: E—is a main view of the actual LED photoreactor device setup with typical arrangement of the photochemical reaction.

Figure: F—is a main view of the actual blue LED photoreactor device setup with typical arrangement of the photochemical reaction.

Figure: G—is a top view, side view and back view of the actual LED photoreactor device setup with typical arrangement of the photochemical reaction.

### DETAILED DESCRIPTION OF THE INVENTION

The above and similar other objectives of the present invention are achieved by the arrangement of the photochemical reactor assembly and developed methods for performing the photochemical transformations using the instantly presented device.

In general, the various terms used herein pertaining to the instantly presented invention are defined herein below:

As used herein, the term “light source” refers to a source of light which is essential for the initiation of the photocatalytic transformations. In particular, the device of the instant invention consists of light emitting diode (LED) as source of light. The LEDs are also termed as solid-state lighting” (SSL) or solid-state lighting source. The LEDs are p-n junction diodes, acting as semiconductor devices, which are best driven by direct current. The brightness of the LEDs depends on the current flowing through the LED. Therefore, increasing current supplied to an LED increases the brightness of the LED and decreasing current supplied to the LED downs the LED brightness. The LEDs for use with the present invention typically have significant emission levels of light having wavelengths about 450 nm (nanometers). The wavelength of light is selected such that they sufficiently initiate the photochemical transformation. For the purpose of the instant invention, preferably blue colour emitting LEDs are used. Apart from this, LEDs with different wavelength are also used such as Violet (395-430 nm), Indigo (430-450 nm), Blue (450-480 nm), Blue-Green (480-520 nm), Green (520-555 nm), Yellow-Green (555-585 nm), Yellow (585-600 nm), Amber (600-615 nm), Orange (615-625 nm), Orange-Red (625-640 nm) and/or Red (640-700 nm).

As used herein, the term “heat sink” refers to a thermal conductive metal device designed to absorb and disperse heat away from a high temperature object, eventually increases the efficiency of the assembly. In general, the heat sinks are outfitted with light source panel, to maintain the appropriate temperature. All in all the, heat sinks are actually performing the heat dissipation mechanism, contributing in controlling the reaction temperature. For the purpose of the instant invention, the heat sinks are made out of metal, such as aluminium, and are attached to the LED panels. The heat sinks have fins, as thin slices of metal connected to the base of the heat sink, which help spread heat over a large area.

As used herein, the term “cooling assembly” refers to a cooling fan that enhances the cooling mechanism which in-turn contributing in the heat dissipation of the assembly. The use of cooling fans which are placed below the aluminium panel heat sink assembly exhaust the heat out from the system, helping in controlling the reaction temperature. The cooling fan used in the instantly proposed photoreactor assembly may be similar to the CPU cooling fans. The combination of a heat sink and fan is referred to as an active heat sink, while a heat sink without a fan is a passive heat sink.

Accordingly, the present invention relates to a photochemical rector device as shown in fig. A-G; comprising of:

(a) light source (1),

(b) heat dissipation assembly (3) and

(c) cooling assembly (4).

In an embodiment, the light source is selected from the group consisting of light emitting diode (LED).

In an embodiment, the heat dissipation assembly is selected from the group consisting of Aluminium based heat sink.

In an embodiment, the cooling assembly is selected from the group consisting of cooling fan.

Accordingly, the present invention relates to a photochemical rector assembly device as shown in fig. A-G, comprising of

(i) light emitting diode (LED) panel (1),

(ii) Aluminium based heat sink (3), and

(iii) cooling fan (4).

The photochemical rector assembly device of the instantly presented invention is schematically shown in fig. A-D, comprising of a light source; the preferable light source is light emitting diode (LED) (1). The LEDs are fixed on a panel by any of the regular fixing material, and supplied with the direct current of about 12 volts. Typically, the alternate current (AC) and direct current (DC) supply is controlled by the adapter as shown in the fig. D. The preferable arrangement of LEDs comprises fixing of four LEDs each of 10 watt capacity, which eventually forms 40 watt LED light source panel. In the instant case, blue light emitting LEDs (1) are placed to form the light source panel.

Subsequently, the LED panel is positioned by fixing on the heat dissipation assembly; the preferable heat dissipation assembly is a metal based heat sink, which is aluminium based heat sink (3). It is a thermal conductive aluminium plate designed to absorb and disperse heat, generated due to blue light emitting LEDs (1). The aluminium heat sink is positioned between the LED light source panel and the cooling fan, to maintain the appropriate temperature. The aluminium plate heat sinks have fins, as thin slices of metal connected to the base of the heat sink, which help spread heat over a large area; that further ends to the cooling fan.

Further, the third essential parameter of the instantly presented photochemical rector device is the cooling assembly; the preferable cooling assembly is the cooling fan (4). The cooling fan is positioned below the aluminium panel heat sink assembly (3). The use of cooling fans exhausts the heat out from the system, helping in controlling the reaction temperature. The cooling fan (4) used in the instantly proposed photoreactor assembly may be similar to the CPU cooling fans or routinely used exhaust fans. The unique combination of an aluminium heat sink and fan, below the central LED panel (1) helps in effectively maintain the temperature.

The fig. D depicts the main view of the LED photochemical reactor device with electric circuit represented by the schematic diagram constructed and arranged in accordance with the present invention. The electric circuit as per fig. D indicates that the active LED panel (1) is connected to an adapter, to channelize the AC-DC supply. Through the adapter, the LEDs are supplied with the AC input of about 110-220 volt corresponds to the DC output of about 12 volts.

The fig. B and fig. C depicts the schematic representation of the properly assembled photochemical reactor device as main view and top view respectively, constructed and arranged in accordance with the present invention. The fig B-C represented typical set up of the instantly presented photochemical reactor device. The device is assembled with the light emitting diode (LED) panel (1) at the front and cooling fan (4) at the back. The aluminium based metal heat sink (3) is sandwiched between the LED panel (1) and cooling fan (4).

The fig. E, fig. F and fig. G depict the actual photograph indicating the practical positioning and implementation of the instantly presented photochemical reactor device. Accordingly, fig. E represents the reaction tube wherein the reaction mixture is filled in the reaction tube with a magnetic needle. The reaction tube is placed on the magnetic stirrer and allowed the reaction mixture to stir. The photochemical reactor assembly device is positioned in such a way that the LED panel (1) facing towards the reaction tube. The DC electric supply is passed through LED panel (1) through the adapters; that illuminates the blue light emitting LEDs.

The fig. F depicts the actual photograph indicating the practical positioning of the device while performing the photocatalytic transformation. Essentially, the darkness is maintained surrounding the reaction mixture, the fully illuminated LEDs (1) starts emitting blue light. For the purpose of the instant device the each LED taken of 10 watt capacity, which collectively corresponds to 40 watt of power. The emitted blue light was observed with the effective wavelength of about 450 nm.

The fig. G depicts the actual photograph indicating the front, side and back view respectively of the instantly presented photochemical reactor device. The fig. G clearly shows that the LED panel (1) is fixed on the heat sink aluminium plate (3) using heat sink paste (2) and simply by screw fitting. The front view depicts the parallel arrangement of four IOW blue light emitting diodes (LEDs) (1) connected with the DC electric supply. The side view of the device depicts the critically sandwiched position of the aluminium heat sink (3) between LED panel (1) and cooling fan (4). The side view clearly shows the fins of the aluminium heat sinks (3), as thin slices of metal connected to the base of the heat sink. The base of the metal plate is comparatively thick. The back view of the photochemical reactor assembly clearly depicts the posteriorly positioned cooling fans. The cooling fan eventually helps exhausting out the heat.

In an embodiment, the present invention relates to an improved photochemical transformation reaction by exposing the reaction mixture to a photochemical rector device as shown in fig. A-G, comprising of a light source (1), a heat dissipation assembly (3) and a cooling assembly (4).

In yet another embodiment, the present invention relates to an improved photochemical transformation reaction by exposing the reaction mixture to a photochemical rector device as shown in fig. A-G, comprising of (i) light emitting diode (LED) panel (1) (as described herein), (ii) Aluminium based heat sink (3) (as described herein), and (iii) cooling fan (4) (as described herein).

Accordingly, the present invention relates to an improved photochemical transformation reaction by exposing the reaction mixture to a photochemical rector device as shown in fig. A-G, comprising of:

(i) light emitting diode (LED) panel (1),

(ii) Aluminium based heat sink (3), and

(iii) cooling fan (4).

In an embodiment, the preferable arrangement of LEDs comprises fixing of four LEDs each of 10 watt capacity, which eventually forms 40 watt LED light source panel. In the instant case, blue light emitting LEDs are placed to form the light source panel.

In yet another embodiment, there is provided an improved photocatalytic -4 (Carbon-Carbon) coupling reaction by exposing the reaction mixture to a photochemical rector device as shown in fig. A-G, comprising of (i) light emitting diode (LED) panel (1), (ii) Aluminium based heat sink (3), and (iii) cooling fan (4).

It is evident from the literature documents such as J. Am. Chem. Soc.138 (26), pp 8084-8087 (2016) disclosed the photocatalytic sp3-sp2 Carbon-Carbon couplings using Blue LED light comprising silyl radical activation of alkyl halides in metallaphotoredox catalysis. Similarly, the reference Science 353(6296): p279-83 (2016) disclosed the photocatalytic C—N coupling reaction aryl amination using ligand-free Ni(II) salts and photoredox catalysis.

In an embodiment, the reactions of the present invention are carried out in presence of a photocatalyst such as [4,4′-Bis(tert-butyl)-2,2′-bipyridine]bis[3,5-difluoro-2-[5-(trifluoromethyl)-2-pyridinyl]phenyl]iridium(III)hexafluorophosphate (Ir[dF(CF₃)PPY]₂(dtbbpy)PF₆).

The photoreactor of the present invention is extended to use in the process comprising the photocatalytic sp3-sp2 Carbon-Carbon coupling and it is illustrated in the following Scheme-I,

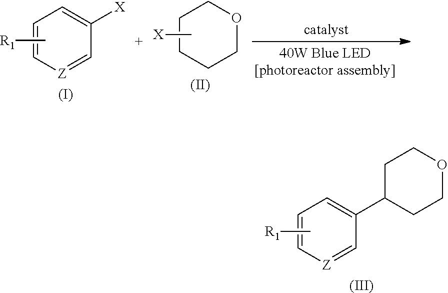

wherein, ‘R₁’ is selected from hydrogen, alkyl, cyclo-alkyl, carbonyl, ester, ether, aryl or hetero-aryl;

‘X’ is halo selected from F, Cl, Br or I:

‘Z’ is selected from C, N, S.

The process of the present invention as illustrated in the above Scheme-I comprise reaction of the aryl compound (1) with the cyclo compound (II) in the presence of catalyst and exposed to the instantly presented photoreactor assembly consisting of 40 W blue LED panel and cooling assembly, to provide sp3-sp2 Carbon-Carbon coupled compound (III) with about 83% yield and a purity of at least 81% LCMS purity.

Similarly, the photoreactor of the present invention is extended to use in the process comprising the photocatalytic Buchwald type (C-N) coupling and it is illustrated in the following Scheme-II,

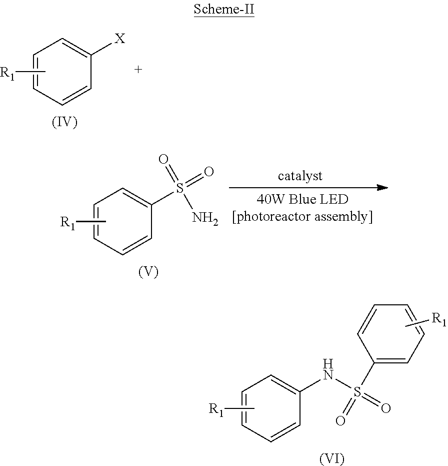

wherein, ‘R₁’ is selected from hydrogen, alkyl, cyclo-alkyl, carbonyl, ester, ether, aryl or hetero-aryl;

‘X’ is halo selected from F, Cl, Br or I;

‘Z’ is selected from C, N, S

The process of the present invention as illustrated in the above Scheme-II comprise reaction of the aryl compound (IV) with the compound (V) in the presence of catalyst and exposed to the instantly presented photoreactor assembly consisting of 40 W blue

LED panel and cooling assembly, to provide C-N coupled compound (VI) with about 74% yield and a purity of at least 99% LCMS purity.

Also, the photoreactor of the present invention is extended to use in the process comprising the photocatalytic radical addition of vinyl boronates and it is illustrated in the following Scheme-III,

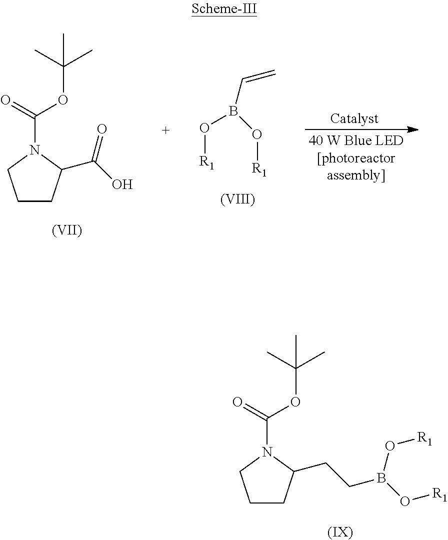

wherein, ‘R₁’ is selected from hydrogen, alkyl, cyclo-alkyl, carbonyl, ester, ether, aryl or hetero-aryl;

The process of the present invention as illustrated in the above Scheme-HI comprise reaction of the carboxyl compound (VII) with the compound (VIII) in the presence of catalyst and exposed to the instantly presented photoreactor assembly consisting of 40W blue LED panel and cooling assembly, to provide C-N coupled compound (IX) with about 52% yield and a purity of at least 72% LCMS purity.

In an embodiment, the aluminium heat sink provides a medium to dissipate the heat produced during the operation of the assembly. The cooling fan speeds up the dissipation of the heat generated and this leads to the lesser impurity profile.

Inventors of the instantly presented photochemical reactor assembly have observed that when the photochemical reactions were carried out previously by using commercially available LED strips that were fixed inside the wall of a regular crystallization dish: the reactions were not clean due to extended reaction time as well as lower wattage (12 W) of the LED assembly. The extended reaction time produces heat and in turn the reactions become less clean in terms of yield and impurity profile. Evidently, the current assembly furnishes higher wattage (preferably 40 W) which helps to reduce the reaction time and the in-built fan cools the reaction mixture in such a way that the undesired side products are substantially minimal. This amounts to a significant advantage over the processes reported in the prior art.

Advantageously, the above identified elements of instantly presented improved photochemical rector assembly device, particularly a light emitting diode (LED) based small photochemical reactor and methods for performing the photochemical transformations using the instantly presented device effectively contribute to the reduction of overall cost of the process.

The instantly presented LED based small photochemical reactor device and/or experiments indicting the practical implementation of the device disclosed herein is applicable to but not limited to the below chemistry attributes such as physical process, chemical reaction corresponding to the inter-conversion of organic and/or inorganic, essentially includes a chemical, physical and/or biological process or reaction favored in the presence of light of any wavelength, that is photochemical reactions, such as photosensitized, photoinitiated, photoactivated, photocatalytic or photosynthetic. Similarly, other non-limiting light-assisted reactions of interest includes photoionozation, photoisomerizations, rearrangements, photoreductions, cyclizations, cycloadditions, sigmatropic shifts, photooxidation, photocleavage of protecting groups or linkers, photohalogenations, photosulfochlorinations, photosulfoxidations, photopolymerizations, photonitrosations, photodecarboxylations, decomposition of azo-compounds, reactions such as Buchwald type C—N couplings, sp3-sp2 C—C coupling, Norrish type reactions and Barton type reactions.

Additionally, the device and/or experiments indicting the practical implementation of the device disclosed herein is applicable to but not limited to the below chemistry reactions such as oxidation; reduction; substitution; elimination; addition; ligand exchange; metal exchange; and ion exchange. Further, the instantly presented LED based small photochemical reactor device and/or experiments indicting the practical implementation of the device disclosed herein is applicable to but not limited to the below chemistry reactions such as polymerisation; alkylation; dealkylation; nitration; peroxidation; sulfoxidation epoxidation; ammoxidation; hydrogenation; dehydrogenation; organometallic reactions; precious metal chemistry/homogeneous catalyst reactions; carbonylation: thiocarbonylation; alkoxylation; halogenation: dehydrohalogenation; dehalogenation: hydroformylation; carboxylation; decarboxylation; amination; arylation; peptide coupling; condensations; cyclocondensation; dehydrocyclization; esterification; amination; heterocyclic synthesis; dehydration; alcoholysis; hydrolysis; ammonolysis; etherification; enzymatic synthesis; ketalization; saponification; isomerisation; formylation; metathesis; silylations; nitrile synthesis; phosphorylation; ozonolysis; azide chemistry; enzymatic reactions; hydrosilylation; coupling reactions; and/or phase transfer reactions.

Additionally, the critical heat dissipation assembly and cooling fan, are connected to the LED panel, it helps conducive heat dissipation from the LED light source, and also facilitate the smooth discharge of hot air from the reaction as well as heat from light source. That helps to function the LED lamp for longer time by controlling the lower reaction temperature, that increase overall service life of LED bulb and also increases the stability of the light source.

The invention is further illustrated by the following examples which are provided to be exemplary of the invention, and do not limit the scope of the invention. While the present invention has been described in terms of its specific embodiments, certain modifications and equivalents will be apparent to those skilled in the art and are intended to be included within the scope of the present invention.

### EXAMPLES

### Example-1

### Preparation of methyl 4-(tetrahydro-2H-pyran-4-yl)benzoate (III¹)

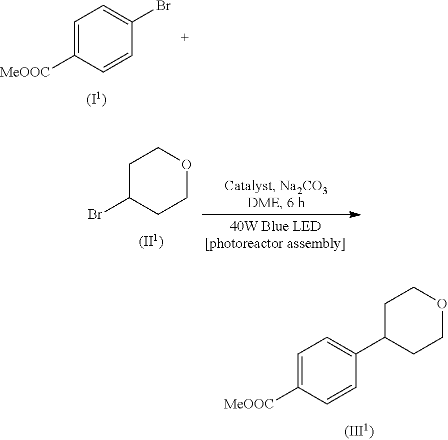 [SMILES: COC(=O)c1ccc(Br)cc1 ; BrC1CCOCC1 ; COC(=O)c1ccc(C2CCOCC2)cc1]

Charged 8.0 mL of 1,2-Dimethoxyethane (DME) in a reaction vial followed by the addition of photocatalyst Ir[dF(CF₃)ppy]₂(dtbbpy)PF₆ (10 mg, 9.3 μmol, 0.01 equiv.), methyl 4-bromo benzoate (I¹) (0.2 g, 0.93 mmol, 1.0 equiv.), 4-bromotetrahydropyran (II¹) (0.23 g, 1.395 mmol, 1.5 equiv.), tris(trimethylsilyl)silane (0.231 g, 0.93 mmol, 1.0 equiv) and anhydrous sodium carbonate (0.197 g, 1.86 mmol, 2.0 equiv.) under nitrogen atmosphere. In another reaction vial, charged 2.0 mL of DME followed by the addition of NiCl₂ glyme (2.0 mg, 9.3 μmol, 0.01 equiv.) and 4,4′-di-tert-butyl-2,2′-bipyridine (2.4 mg, 9.3 pmol, 0.01 equiv.) under nitrogen atmosphere. The reaction mixture of second vial was stirred for 10 minutes and added slowly to the reaction mixture of first vial. The reaction mixture was sealed with blue septa and exposed to the photochemical reactor assembly (fig. A-G). The reaction mixture was irradiated with 40 W Blue LED while stirring for 6 hours. The reaction mixture was poured into water and extracted with ethyl acetate. The combined organic layer was washed with brine, dried over S sodium sulphate and concentrated under vacuum to afford a crude residue of product compound (1115. The crude product was further purified by Reverse Phase combi flash (0-59% acetonitrile in 0.1% FA in water) to obtain the purified desired product compound-(III') (0.17 g, 83% yield, 81% pure by LCMS).

### Example-2

### Preparation of 3-(tetrahydro-2H-pyran-4-yl)pyridine (III²):

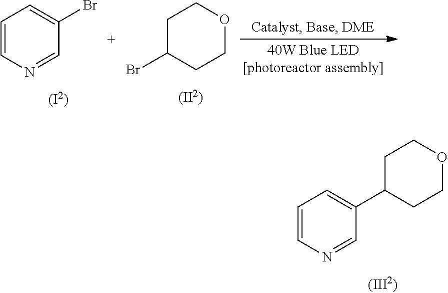 [SMILES: Brc1cccnc1 ; BrC1CCOCC1 ; c1cncc(C2CCOCC2)c1]

Charged 3.0 mL of 1,2-Dimethoxyethane (DME) in a reaction vial followed by the addition of photocatalyst Ir[dF(CF₃)ppy]₂(dtbbpy)PF₆ (7 mg, 0.0063 mmol, 0.01 equiv.), 3-bromo pyridine (I²) (0.1 g, 0.632 mmol, 1.0 equiv.), 4-bromotetrahydropyran (II²) (0.156 g, 0.949 mmol, 1.5 equiv.), tris(trimethylsilyl)silane (0.157 g, 0.632 mmol, 1.0 equiv) and anhydrous lithium hydroxide (0.03 g, 1.265 mmol, 2.0 equiv.) under nitrogen atmosphere. In another reaction vial, charged 1.0 mL of DME followed by the addition of NiCl₂ glyme (1.3 mg, 0.0063 mmol, 0.01 equiv.) and 4,4′-di-tert-butyl-2,2′-bipyridine (1.6 mg, 0.0063 mmol, 0.01 equiv.) under nitrogen atmosphere. The reaction mixture of second vial was stirred for 10 minutes and added slowly to the reaction mixture of first vial. The reaction mixture was sealed with blue septa and exposed to the photochemical reactor assembly (fig. A-G). The reaction mixture was irradiated with 40 W Blue LED while stirring for 16 hours. The reaction mixture was poured into water and extracted with ethyl acetate. The combined organic layer was washed with brine, dried over sodium sulphate and concentrated under vacuum to afford a crude residue of product compound (III²).

The crude product was further purified by 60-120 mesh silica gel column chromatography (20-80% Ethyl-acetate in Hexane) to obtain the purified desired product compound-(III²) (0.095 g, 92% yield, 87% pure by LCMS).

### Example-3

### Preparation of methyl 4-(phenylsulfonamido)benzoate (VI¹):

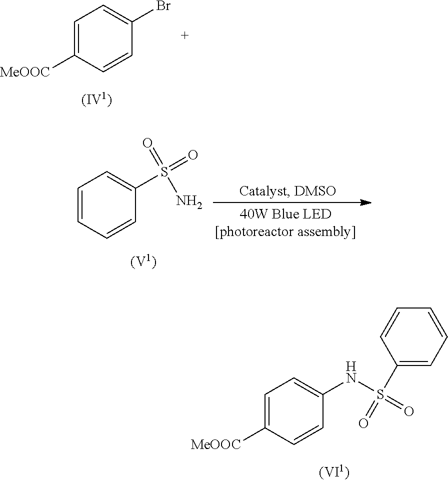 [SMILES: COC(=O)c1ccc(Br)cc1 ; NS(=O)(=O)c1ccccc1 ; COC(=O)c1ccc(NS(=O)(=O)c2ccccc2)cc1]

10

Charged 5.0 mL of dimethylsulfoxide (DMSO) in a reaction vial followed by the addition of photocatalyst Ir[dF(CF₃)ppy]₂(dtbbpy)PF₆ (2 mg, 0.0023 mmol, 0.005 equiv.), methyl 4-bromo benzoate (IV¹) (0.1 g, 0.465 mmol, 1.0 equiv.), benzenesulfonamide (V¹) (0.109 g, 0.697 mmol, 1.5 equiv.), NiCl₂ glyme (5 mg, 0.023 mmol, 0.05 equiv.), 4,4′-di-tert-butyl-2,2′-bipyridine (1.2 mg, 0.0046 mmol, 0.01 equiv.) and 1,1,3,3-Tetramethylguanidine (0.08 g, 0.697 mmol, 1.5 equiv.). The reaction mixture was sealed with blue septa and exposed to the photochemical reactor assembly (fig. A-G). The reaction mixture was irradiated with 40 W Blue LED while stirring for 16 hours. The reaction mixture was poured into water and extracted with ethyl acetate. The combined organic layer was washed with brine, dried over sodium sulphate and concentrated under vacuum to afford a crude residue of product compound (VI¹). The crude product was further purified by 60-120 mesh silica gel column chromatography (0-15% Ethyl-acetate in Hexane) to obtain the purified desired product compound-(VI¹) (0.1 g, 74% yield, 99% pure by LCMS).

### Example-4

### Preparation of tert-butyl 2-(2-(4,4,5,5-tetramethyl-1,3,2-dioxaborolan-2-yl)ethyl)pyrrolidine-1-carboxylate (IX')

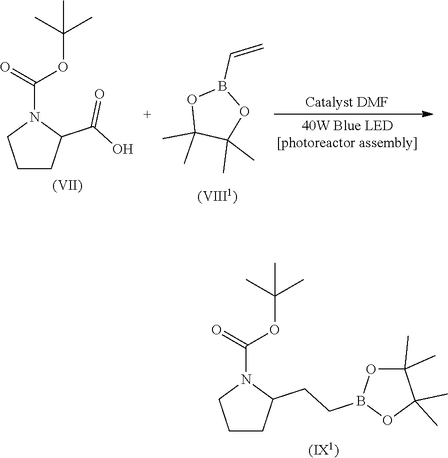

Charged 5.0 mL of dimethylformarnide (DMF) in a reaction vial followed by the addition of photocatalyst Ir[dF(CF₃)ppy]₂(dtbbpy)PF₆ (4 mg, 0.0046 mmol, 0.01 equiv.), (tert-butoxycarbonyl)proline (VII) (0.1 g, 0.465 mmol, 1.0 equiv.), 4,4,5,5-tetramethyl-2-vinyl-1,3,2-dioxaborolane (VIII) (0.107 g, 0.697 mmol, 1.5 equiv.), and Cs₂CO₃ (0.166 g, 0.512 mmol, 1.1 equiv.). The reaction mixture was sealed with blue septa and exposed to the photochemical reactor assembly (fig.A-G). The reaction mixture was irradiated with 40 W Blue LED while stirring for 20 hours. The reaction mixture was poured into water and extracted with ethyl acetate. The combined organic layer was washed with brine, dried over sodium sulphate and concentrated under vacuum to afford a crude residue of product compound (IX¹). The crude product was further purified by Reverse Phase combi-flash (0-70% acetonitrile in water) to obtain the purified desired product compound-(IX¹) (0.08 g, 52% yield, 72% pure by LCMS).

1. A photochemical rector device assembly as shown in Figures: A-G;
comprising of
(a) light source (1);
(b) heat dissipation assembly (3); and
(c) cooling assembly (4).

2. The device according to the claim 1, wherein the light source (1) is a light emitting diode (LED) panel having wavelength selected from Violet (395-430 nm), Indigo (430-450 nm), Blue (450-480 nm), Blue-Green (480-520 nm), Green (520-555 nm), Yellow-Green (555-585 nm), Yellow (585-600 nm), Amber (600-615 nm), Orange (615-625 nm), Orange-Red (625-640 nm) and/or Red (640-700 nm).

3. The device according to the claim 1, wherein the heat dissipation assembly (3) is an aluminium based heat sink.

4. The device according to the claim 1, wherein the cooling assembly (4) is a cooling fan.

5. A process for the preparation of compound (III) of the following formula, 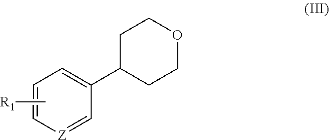
comprising of the, photocatalytic sp³-sp² Carbon-Carbon coupling reaction of the compound (I) and compound (II) of the following formula, 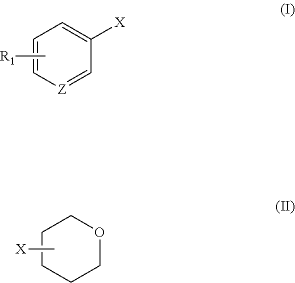
wherein, ‘R₁’ is selected from hydrogen, alkyl, cyclo-alkyl, carbonyl, ester, ether, aryl or hetero-aryl; ‘X’ is halo selected from F, Cl, Br or I; ‘Z’ is selected from C, N, S.
wherein, the reaction is carried out in the presence of a catalyst and using the photochemical rector device assembly of claim 1 as shown in Figure: A-G.

6. A process for the preparation of compound (VI) of the following formula, 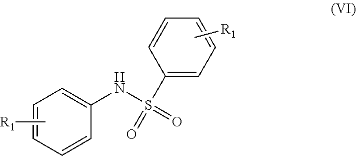
comprising of the, photocatalytic Buchwald type (C-N) coupling reaction of the compound (IV) and compound (V) of the following formula, 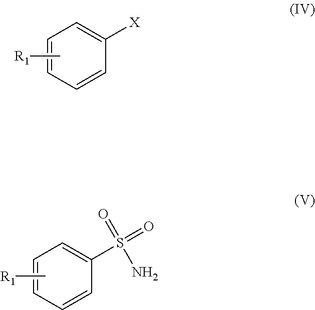
wherein, ‘R₁’ is selected from hydrogen, alkyl, cyclo-alkyl, carbonyl, ester, ether, aryl or hetero-aryl; ‘X’ is halo selected from F, Cl, Br or I; ‘Z’ is selected from C, N, S.
wherein, the reaction is carried in the presence of a catalyst and using the photochemical rector device assembly of claim 1 as shown in Figure: A-G. (Original) A process for the preparation of compound (IX) of the following formula, 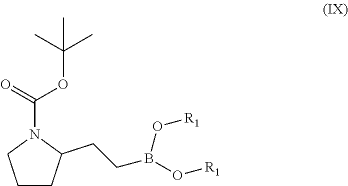
comprising of the, photocatalytic radical addition reaction of vinyl boronates, of the compound (IV) and compound (V) of the following formula, 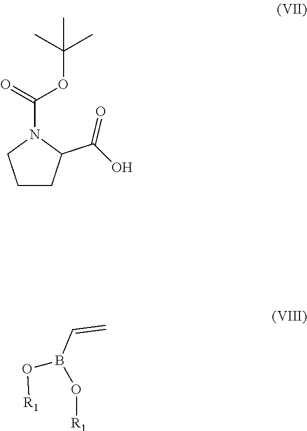
wherein, ‘R₁’ is selected from hydrogen, alkyl, cyclo-alkyl, carbonyl, ester, ether, aryl or hetero-aryl;
wherein, the reaction is carried in the presence of a catalyst and using the photochemical rector device assembly of claim 1 as shown in Figure: A-G.

8. The process according to claim 5, wherein the catalyst is a photocatalyst selected from [4,4′-Bis(tert-butyl)-2,2′-bipyridine]bis[3,5-difluoro-2-[5-(trifluoromethyl)-2-pyridinyl]phenyl]iridium(III) hexafluorophosphate (Ir[dF(CF₃)ppy]₂ (dtbbpy)PF6).

9. The process according to 6, wherein the catalyst is a photocatalyst selected from [4,4′-Bis(tert-butyl)-2,2′-bipyridine]bis[3,5-difluoro-2-[5-(trifluoromethyl)-2-pyridinyl] phenyl]iridium(III) hexafluorophosphate (Ir[dF(CF₃)ppy]2(dtbbpy)PF6).

10. The process according to 7, wherein the catalyst is a photocatalyst selected from [4,4′-Bis(tert-butyl)-2,2′-bipyridine]bis[3,5-difluoro-2-[5-(trifluoromethyl)-2-pyridinyl] phenyl]iridium(III) hexafluorophosphate (Ir[dF(CF₃)ppy]2(dtbbpy)PF6).
